(19) United States
(12) Patent Application Publication
(10) Pub. No.: US 2003/0114802 A1
(43) Pub. Date: Jun. 19, 2003

(54) Flow regulator for IV tubing set

(76) Inventors: Chien-Wei Chung, Taichung (TW); Chin-Shu Huang, Hsinchu (TW); Tzu-Sheng Fan, Miao Li Hsien (TW)

Correspondence Address: BROWDY AND NEIMARK, P.L.L.C., 624 NINTH STREET, NW, SUITE 300, WASHINGTON, DC, 20001-5303, US

(21) Appl. No.: 10/318,195

(22) Filed: Dec. 13, 2002

(30) Foreign Application Priority Data
Dec. 13, 2001 (TW) 90221792

(51) Int. Cl. A61M 5/00

(52) U.S. Cl. 604/250

(57) ABSTRACT

A flow regulator is connectable to an infusion tube of an IV tubing set which having a needle holder and an infusion needle. The flow regulator comprises a housing having an body, a through hole longitudinally extended through front and rear sides of the body for the passing of the infusion tube, a regulation controller installed in the housing and adapted to apply pressure to the infusion tube extended through the through hole of the housing, and a receiving barrel having an body fixedly fastened to the housing, a receiving chamber defined in the body of the receiving barrel and adapted to receive the infusion needle of the IV tubing set with which the flow regulator is used, and an opening in one end of the body of the receiving barrel through which the infusion needle of the IV tubing set is inserted into the receiving chamber.

### BACKGROUND OF THE INVENTION

The present invention relates to medical instrument and, more specifically, to a flow regulator for use with a IV tubing set to regulate liquid flow passing through an infusion tube of the IV tubing set.

After the service of an IV tubing set, the infusion needle must be well packed or protected against contamination. Normally, a protective cap is used and capped on the infusion needle after the service of the IV tubing set. There is known IV tubing set having a slide sleeve coupled to the infusion tube and adapted for receiving the needle assembly of the IV tubing set after the service of the IV tubing set. This design is not convenient in use. When received in the sliding sleeve. In either of the aforesaid conventional infusion needle protective methods, the infusion needle may be forced out of protection accidentally, resulting in an accident.

### SUMMARY OF THE INVENTION

The present invention has been accomplished under the circumstances in view.

It is the main object of the present invention to provide a flow regulator for IV tubing set, which has means to receive the infusion needle after the service of the IV tubing set.

It is another object of the present invention to provide a flow regulator for IV tubing set, which is easy and safe in use.

It is still another object of the present invention to provide a flow regulator for IV tubing set, which is inexpensive to manufacture and easy to install.

To achieve these and other objects of the present invention, the flow regulator is connectable to the infusion tube of a IV tubing set for adjusting liquid flow through the infusion tube, which IV tubing set having a needle holder and an infusion needle at one end of the infusion tube. The flow regulator comprises a housing, the housing comprising an body, a through hole longitudinally extended through front and rear sides of the body for the passing of the infusion tube, a regulation controller installed in the housing and adapted to apply pressure to the infusion tube extended through the through hole of the housing, and a receiving barrel, the receiving barrel comprising an body fixedly fastened to the housing, a receiving chamber defined in the body of the receiving barrel and adapted to receive the infusion needle of the IV tubing set with which the flow regulator is used, and an opening in one end of the body of the receiving barrel through which the infusion needle of the IV tubing set is inserted into the receiving chamber. After the service of the IV tubing set, the infusion needle of the IV tubing set is received in the receiving chamber inside the receiving barrel, prohibiting a contamination.

For better use, the receiving barrel further comprises retaining means adapted for engaging the needle holder to secure the infusion needle in the receiving chamber.

### BRIEF DESCRIPTION OF THE DRAWINGS

FIG. 1 is side view in section of the first embodiment of the present invention.

FIG. 2 is similar to FIG. 1 but showing the infusion needle received in the receiving chamber inside the receiving barrel.

FIG. 3 is a side view in section of an alternate form of the present invention, showing the receiving barrel provided with a hinged sealing cap.

FIG. 4 is an elevational view of another alternate form the present invention.

FIG. 5 is a side view in section of the embodiment shown in FIG. 4.

FIG. 6 is similar to FIG. 6 but showing the infusion needle received in the receiving chamber of the receiving barrel.

FIG. 7 is a side plain view of still another alternate form of the present invention.

FIG. 8 is a side plain view of still another alternate form of the present invention.

### DETAILED DESCRIPTION OF THE INVENTION

Referring to FIG. 1, a flow regulator with infusion needle receiving means is connectable to the infusion tube 6 of a IV tubing set for regulating the flow rate of the infusion solution and receiving the needle assembly at one end of the infusion tube 6.

The needle assembly of the IV tubing set comprises a needle holder 7 and an infusion needle 8. The needle holder 7 is fastened to one end of the infusion tube 6, having a first retaining means 71 which formed of four beveled guide ribs equiangularly spaced around the periphery and a second retaining means 73 which formed of an annular flange extended around the periphery and spaced between the first retaining means 71 and the infusion needle 8. The other end of the infusion tube 6 is connected to the solution bottle (not shown) of the IV tubing set.

Referring to FIG. 1 again, the flow regulator comprises a housing 10, a regulation controller 20, and a receiving barrel 30.

The housing 10 comprises an body 11, a through hole 12 longitudinally extended through front and rear sides of the body 11, a mount 13 integral with one side of the body 11, two sliding grooves 14 formed in the mount 13 and disposed in a sloping position relative to the through hole 12.

The regulation controller 20 is installed in the mount 13 of the housing 10, and formed by a wheel 21 and two round rods 23 at two sides of the wheel 21. The round rods 23 are slidably mounted in the sliding grooves 14 of the housing 10. The wheel 21 has a serrated periphery 22 adapted to apply pressure evenly across the entire width of the infusion tube 6.

The receiving barrel 30 comprises an body 31 integral with one side of the housing 10, a front opening 33 in one end of the body 31, a rear opening 34 in the other end of the body 31, a receiving chamber 32 connected between the front opening 33 and the rear opening 34, and a retaining portion 35 integral with the body 31 around the rear opening 34.

When installed, the infusion tube 6 movably extends through the rear opening 34, the receiving chamber 32 and the front opening 33, and the needle holder 7 with the infusion needle 8 are disposed outside the front opening 33.

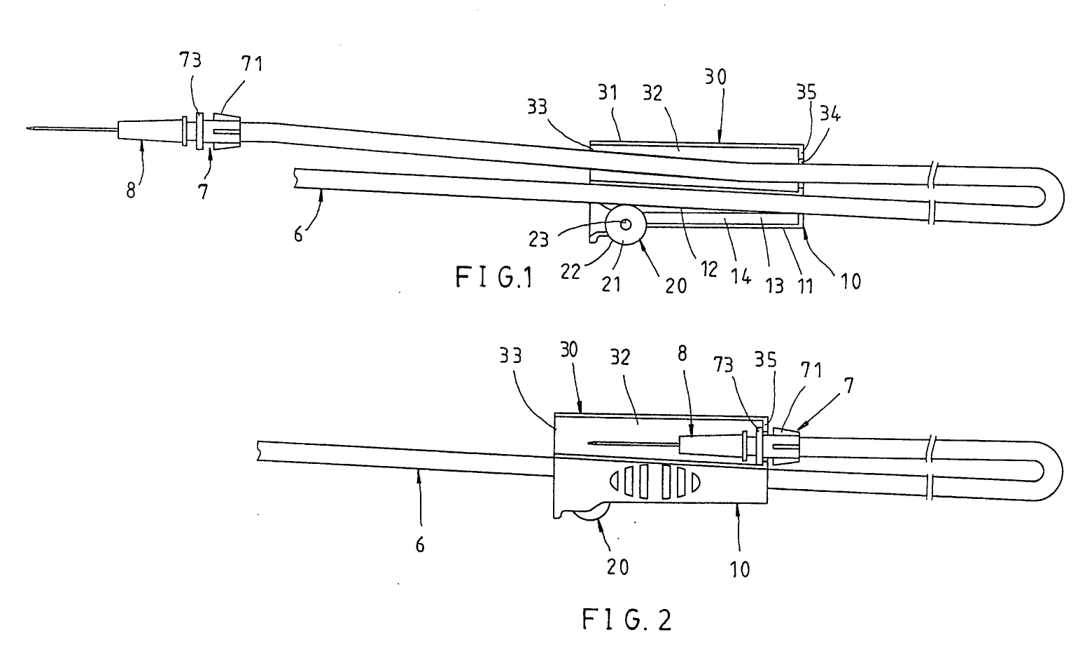

Referring to FIG. 2, when pulling the infusion tube 6 backwards relative to the receiving barrel 30, the needle holder 7 and the infusion needle 8 are moved backwards with the infusion tube 6 through the front opening 33 into the receiving chamber 32. When continuously pulling the infusion tube 6 backwards after the needle holder 7 entered the receiving chamber 32, the first retaining means 71 are forced to pass through the front opening 34 to the outside of the barrel 30, thereby causing the retaining portion 35 to be engaged in between the beveled guide rids 71 and the second retaining means 73 to hold the infusion needle 8 positively in the receiving chamber 32.

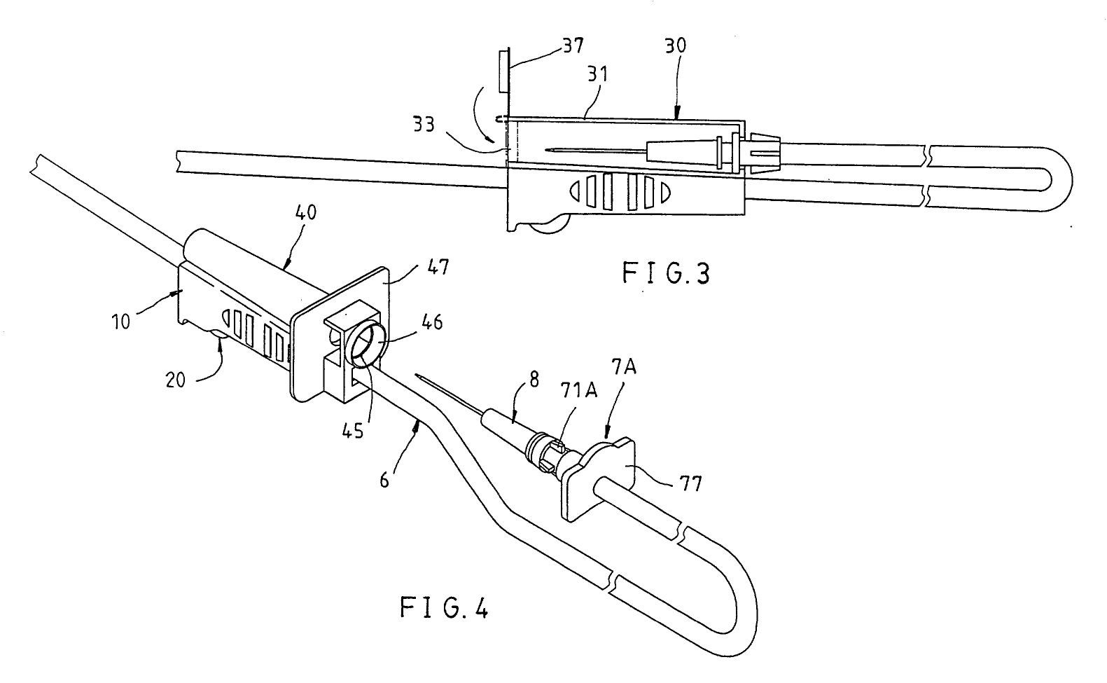

Referring to FIG. 3, a seal cap 37 is hinged to the body 31 of the receiving barrel 30 and adapted for closing the front opening 33 after the infusion needle has been received inside the receiving chamber of the receiving barrel 30.

FIGS. 4˜6 show an alternate form of the present invention. The needle holder 7A of the IV tubing set with which the flow regulator is to be used has a first retaining means 71A which formed of four beveled guide ribs equiangularly spaced around the periphery, and a finger guard 77 at the rear side for protecting the fingers when receiving the infusion needle 8.

According to this embodiment, the flow regulator comprises a holder 10, a regulation controller 20, and a receiving barrel 40.

The receiving barrel 40 comprises a body 41, a receiving chamber 42 formed in the body 41, an opening 43 in one end of the receiving chamber 42, a retainer 45 suspended outside the opening 43, a tapered insertion hole 46 extended through the retainer 45 and aimed at the opening 43, and a retaining hole 44 defined between the tapered insertion hole 46 and the opening 43, and a stop plate 47 extended around the opening 43.

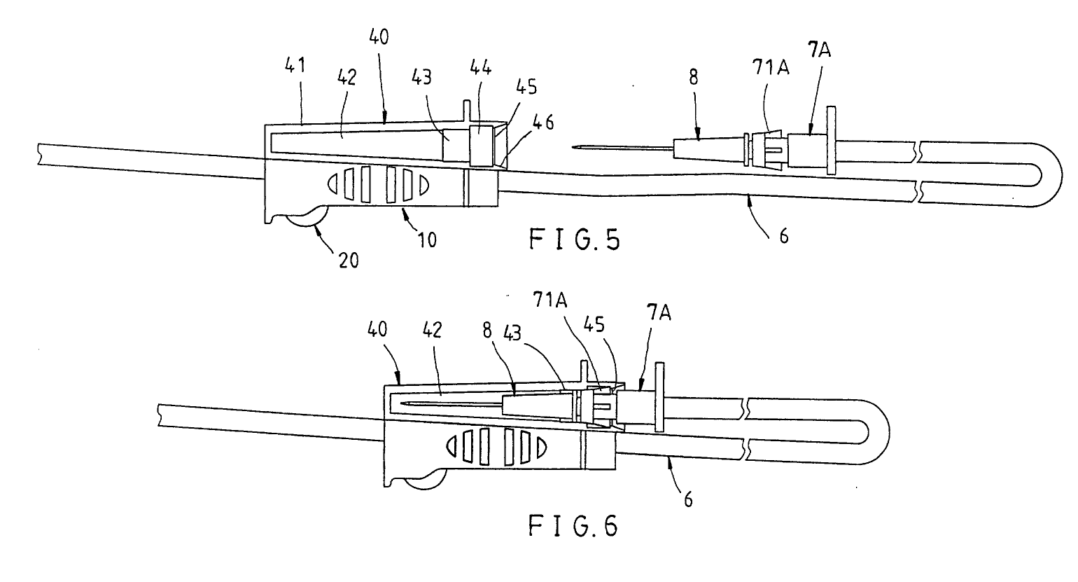

Referring to FIGS. 5 and 6 again, when receiving the infusion needle 8, hold the needle holder 7A with the hand and then insert the infusion needle 8 through the tapered insertion hole 46 and the opening 43 into the receiving chamber 42 to force the first retaining means 71A through the tapered insertion hole 46 into the retaining hole 44. When set into position, the first retaining means 71A are stopped against the inner side of the retainer 45, prohibiting backward movement of the needle holder 7A relative to the receiving barrel 40.

According to the present invention, the housing 10 and the regulation controller 20 can be separately made, and then fastened together by a high-frequency heat sealing apparatus, bonding agent, or joint.

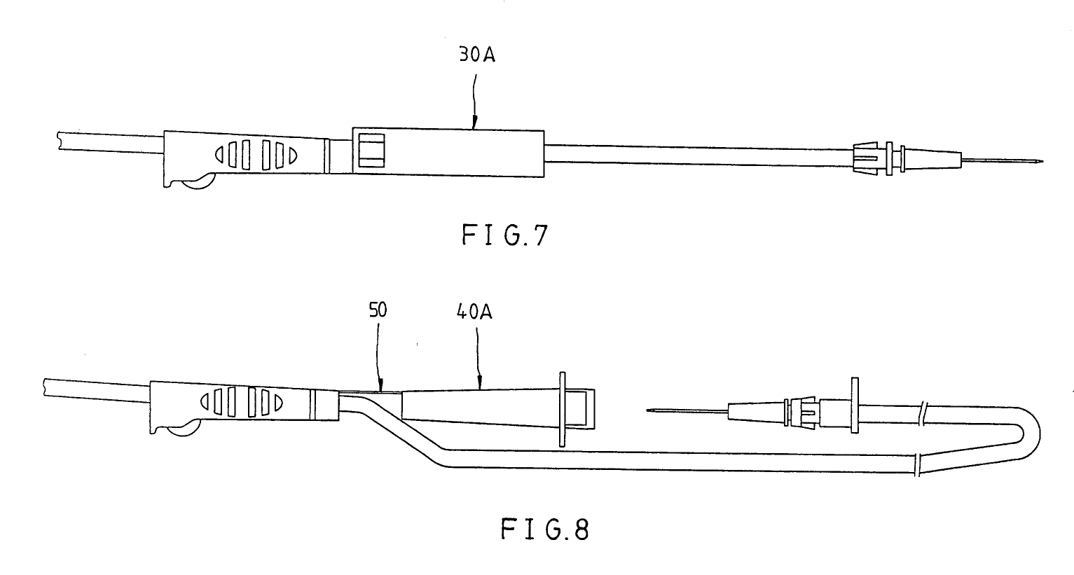

FIG. 7 shows another alternate form of the present invention. According to this embodiment, the receiving barrel 30A is fixedly connected to one end of the housing of the regulation controller in a line.

FIG. 8 shows still another alternate form of the present invention. According to this embodiment, the receiving barrel 40A has a connecting tube 50 fixedly connected to one end of the housing of the regulation controller in a line.

As indicated above, the present invention has advantages as follows:

1. The housing and the receiving barrel can be connected in integrity to reduce the manufacturing and inventory cost.

2. The design of the housing and the receiving barrel enables the user to receive the infusion needle in the receiving chamber safely and positively.

What is claimed is:

1. A flow regulator connectable to an infusion tube of a IV tubing set for adjusting liquid flow through the infusion tube, the IV tubing set having a needle holder and an infusion needle at one end of the infusion tube, the flow regulator comprising:
a housing, said housing comprising an body, a through hole longitudinally extended through front and rear sides of said body for the passing of the infusion tube;
a regulation controller installed in said housing and adapted to apply pressure to the infusion tube extended through the through hole of said housing; and
a receiving barrel, said receiving barrel comprising an body fixedly fastened to said housing, a receiving chamber defined in the body of said receiving barrel and adapted to receive the infusion needle of the IV tubing set with which the flow regulator is used, and a first opening in one end of the body of said receiving barrel through which the infusion needle of the IV tubing set is inserted into said receiving chamber.

2. The flow regulator as claimed in claim 1, wherein said receiving barrel is disposed at one lateral side of said housing.

3. The flow regulator as claimed in claim 1, wherein said receiving barrel is connected to one end of said housing.

4. The flow regulator as claimed in claim 1, wherein said receiving barrel has a retaining means for engaging a retaining means of the needle holder of the IV tubing set to hold the infusion needle in said receiving chamber.

5. The flow regulator as claimed in claim 4, wherein said retaining means of the receiving barrel is disposed around said first opening.

6. The flow regulator as claimed in claim 4, wherein said retaining means of the receiving barrel has a through hole suspended outside said first opening.

7. The flow regulator as claimed in claim 1, wherein said receiving barrel further comprises a second opening in an opposite end of the body in communication with said receiving chamber for the passing of the infusion tube of the IV tubing set and for stopping the infusion needle of the IV tubing set inside said receiving chamber.

8. The flow regulator as claimed in claim 7, wherein said receiving barrel comprises a retaining portion disposed around said second opening and adapted for stopping the infusion needle of the IV tubing set inside said receiving chamber.

9. The flow regulator as claimed in claim 7, wherein said receiving barrel further comprises a sealing cap hinged thereto and adapted for stopping said first opening.

10. The flow regulator as claimed in claim 1, wherein said receiving barrel further comprises a finger guard.
